(19) United States
(12) Patent Application Publication
(10) Pub. No.: US 2025/0231190 A1
(43) Pub. Date: Jul. 17, 2025

(54) ENHANCED FLUORESCENCE SIGNAL THROUGH THE APPLICATION OF AROMATIC ADDITIVES ONTO THE MICROSCOPY SAMPLE FOR STANDARD FLUORESCENCE, FLUORESCENCE MICROSCOPY AND COMBINED FLUORESCENCE MALDI MICROSCOPY/IMAGING

(71) Applicant: The Johns Hopkins University, Baltimore, MD (US)

(72) Inventors: Ethan YANG, Baltimore, MD (US); Kristine GLUNDE, Baltimore, MD (US); Caitlin TRESSLER, Baltimore, MD (US); Scot Charles KUO, Baltimore, MD (US); Hoku Kahele WEST-FOYLE, Baltimore, MD (US); Xinyi Elaine SHEN, Baltimore, MD (US)

(21) Appl. No.: 18/854,736

(22) PCT Filed: Apr. 19, 2023
(86) PCT No.: PCT/US2023/019162

§ 371 Date: Oct. 7, 2024

Related U.S. Application Data
(60) Provisional application No. 63/332,457, filed on Apr. 19, 2022.

(51) Int. Cl. G01N 33/68; G01N 1/28; G01N 1/42; G01N 21/64; G01N 33/92; G02B 21/34

(52) U.S. Cl. CPC G01N 33/6851; G01N 1/42; G01N 21/6428; G01N 21/6458; G01N 33/92; G02B 21/34; G01N 2001/2873

(57) ABSTRACT

Methods for enhancing a fluorescence intensity of a sample by adding an aromatic compound thereto. Workflows for a combined fluorescence-MALDI microscopy/imaging instrument also are disclosed comprising combining MALDI imaging and fluorescence imaging of the same sample in one sample preparation step. The presently disclosed workflow reduces the sampling time to one workday and minimizes sample degradation.

### BACKGROUND

There is continuing work to improve the imaging of the distribution of fluorescence signals, including their use in identifying important areas of biomedical samples. For example, PubMed has shown over 30k annual publications that are related to “fluorescence” over the past decade (as of 2021 May 17).

Matrix-assisted laser desorption/ionization (MALDI) mass spectrometry imaging, or short MALDI imaging, is a quickly developing field, with MALDI imaging instrumentation having undergone significant improvements toward clinically relevant imaging speeds within the last five years. MALDI imaging is currently making its way into clinical pathology laboratories to help identify the distribution of important molecules in biomedical samples. No workflows to date, however, have considered combining MALDI imaging and fluorescence imaging of the same sample in one sample preparation step.

### SUMMARY

In some aspects, the presently disclosed subject matter provides a method for obtaining a fluorescence image of a sample, the method comprising:
(a) providing a sample;
(b) depositing one or more aromatic additives onto the sample; and
(c) obtaining a fluorescence image of the sample.

In certain aspects, the one or more aromatic additives have an absorbance ranging from about 250 nm to about 500 nm. In particular aspects, the one or more aromatic comprise a matrix-assisted laser desorption/ionization (MALDI) matrix. In more particular aspects, the MALDI matrix is selected from alpha-cyano-4-hydroxycinnamic acid (CHCA), sinapinic acid (SA) ((2E)-3-(4-hydroxy-3,5-dimethoxyphenyl)prop-2-enoic acid), 2,5-dihydroxybenzoic acid (DHB), 3,4-dihydroxycinnamic acid (DHBA), 2-mercaptobenzothiazole (MBT), trans-3-indoleacrylic acid (IAA), 5-chloro-2-mercaptobenzothiazole (CMBT), 2-hydroxyphenylbenzoic acid (HPBA), 2-amino-4-methyl5-nitropyridine (2a4m5n), 2,6-diydroxyacetophenone (DHAP), N-(1-naphthyl)ethylenediamine (NEDC), trihydroxyacetophenone (THAP), ferulic acid (FA) ((2E)-3-(4-hydroxy-3-methoxyphenyl)prop-2-enoic acid), picolinic acid (PA) (pyridine-2-carboxylic acid), 3-hydroxy picolinic acid (HPA) (3-hydroxypyridine-2-carboxylic acid), caffeic acid (CA), 1,5-diaminonaphthalene (DAN), 9-aminoacridine (9-AA), norharmane (nH), nicotinic acid, pyrozinoic acid, vanillic acid, succinic acid, glycerol, urea, tris buffer (pH 7.3), other cinnamic acid derivatives, and combinations thereof.

In certain aspects, the MALDI matrix further comprises a solvent. In particular aspects, the solvent is selected from acetonitrile, water, methanol, ethanol, propanol, acetone, chloroform, N,N-dimethylformamide (DMF), tetrahydrofuran (THF) and combinations thereof.

In certain aspects, the MALDI matrix further comprises a counter ion or acid additive. In particular aspects, the counter ion or acid additive is selected from H₃PO₄, HNO₃, H₂SO₄, HCl, trifluoroacetic acid (TFA), ammonium-based salts, sodium-based salts, lithium-based salts and potassium-based salts. In particular aspects, MALDI matrix application is preceded by salt doping protocols with sodium and ammonium salts used as acetates or chlorides or carbonates or bicarbonates, among others.

In certain aspects, the depositing of the one or more aromatic additives onto the sample is accomplished by manual spraying, dry sieve deposition, robotic spraying, sublimation, or combinations thereof.

In certain aspects, the method further comprises fluorescence microscopy.

In certain aspects, the method further comprises obtaining a MALDI mass spectrum of the sample. In particular aspects, the method further comprises MALDI imaging.

In certain aspects, the sample is snap frozen or heat treated. In certain aspects, the sample comprises a dried sample.

In certain aspects, the method further comprises embedding the sample in an embedding medium, matrix, or resin. In particular aspects, the embedding medium comprises an M-1 embedding medium, an optimal cutting temperature (OCT) embedding medium, agar, gelatin embedding medium, carboxymethyl cellulose (CMC) medium or ice. In certain aspects, the sample is embedded in formalin-fixed paraffin (FFPE) matrix.

In certain aspects, the method further comprises cryo-sectioning the sample. In particular aspects, the sample is cryo-sectioned onto microscopy glass slides, an indium tin oxide (ITO)-coated slide or other conductive slides, including but not limited to gold slides, aluminum oxide slides, steel plates, among others.

In certain aspects, the method further comprises derivatizing the sample.

In certain aspects, the method comprises analyzing one or more intact proteins. In certain aspects, the method comprises analyzing one or more tryptic peptides. In certain aspects, the method comprises analyzing one or more N-glycans, peptides, lipids, metabolites, drug molecules, and drug metabolites in a spatially resolved manner.

In certain aspects the sample is selected from an organism, an organ, a tissue, a cell, or a fruit, a leaf, or other parts of plants.

In certain aspects, a fluorescence signal of the sample with the one or more aromatic compounds deposited thereon is greater than a fluorescence signal of the sample without the one or more aromatic compounds deposited thereon.

In particular aspects, the fluorescence is an autofluorescence intrinsic to the specimen.

In certain aspects, the fluorescence signal can derive from reagents applied to the sample during preparation. Such reagents include fluorescent dyes, as well as reagents synthesized from such dyes that direct fluorescence to molecules or specimen regions of biomedical interest. In particular aspects, aromatic dyes including rhodamine B, Hoechst 33342, fluorescein isothiocyanate (FITC), indocyanine green (ICG), among others.

In certain aspects, the fluorescence signal of the sample is the result of genetic manipulations, both to introduce fluorescence signal and to localize the signal to regions of biomedical interest.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, and FIG. 1D show the presently disclosed experimental workflow for FluoMALDI imaging. (FIG. 1A) A freshly frozen brain tissue section is first cryo-sectioned and affixed to a conductive indium-tin oxide (ITO) microscopy slide. (FIG. 1B) This is followed by MALDI matrix application. The samples are sprayed with matrix and thickness of choice. (FIG. 1C) The prepared sample is first used for (auto) fluorescence experiments and then, upon completion, (FIG. 1D) the same sample is subjected to MALDI MSI measurement. This workflow enables the seamless production of two separate images of a single tissue section without compromising the spectral or spatial quality of the hyperspectral datasets;

FIG. 2 is a comparison of the current and improved fluorescence-MALDI imaging workflow. The presently disclosed workflow moves the matrix deposition step to before autofluorescence (AF) experiments. Numerous advantages are derived from this modification, most notably the improved fluorescence intensity and the reduced sample degradation. This workflow also opens the possibility for a combined fluorescence-MALDI microscopy/imaging instrument. Lastly, workflows to use matrix deposition to enhance fluorescence or AF could also be performed for fluorescence microscopy/imaging applications alone without the use of MALDI microscopy/imaging;

FIG. 3A, FIG. 3B, FIG. 3C, FIG. 3D, FIG. 3E, FIG. 3F, FIG. 3G, FIG. 3H, FIG. 3I, FIG. 3J, and FIG. 3K demonstrate fluorescence enhancement on pink Sharpie using various kinds of matrices and on various kinds of fluorophores using CHCA. (FIG. 3A) From left to right, the columns demonstrate imaging results from optical scans, MALDI data focused on Rhodamine B peak at 443.2 m/z, raw fluorescence images, magnified fluorescence images of sample borders shown by white squares, and amplified fluorescence images. (FIG. 3B) Quantification of raw fluorescence intensities using different matrices. (FIG. 3C) Fold enhancement of each condition normalized to uncoated samples. (FIG. 3D) Enhancement of pink sharpie lines coated with different passes of CHCA, ranging from 0 pass to 24 passes. (FIG. 3E) Fold enhancement of each condition normalized to uncoated samples. (FIG. 3F) Enhancement of different exogenous fluorophores coated with CHCA. (FIG. 3G) Enhancement of different endogenous fluorophores coated with CHCA. (FIG. 3H) Quantification of raw fluorescence intensities of images of exogenous fluorophores demonstrated in part F. (FIG. 3I) Quantification of raw fluorescence intensities of images of endogenous fluorophores demonstrated in part G. (FIG. 3J) Fold enhancement for each exogenous fluorophore. (FIG. 3K) Fold enhancement for each endogenous fluorophore;

FIG. 4A, FIG. 4B, FIG. 4C, and FIG. 4D. Fluorescence enhancement on healthy mouse brain samples using various kinds of matrices. (FIG. 4A) From left to right, the columns demonstrate imaging results from optical scans, fluorescence images using DAPI, GFP and TRITC channels, and MALDI data in negative ion mode focused on 888.75 m/z, which is the peak of PI 38:4 (FIG. 4B) Quantification of raw fluorescence intensities and fold change using different matrices. (FIG. 4C) Autofluorescence enhancement of healthy mouse brain coated with different passes of CHCA, ranging from 0 to 32 passes. (FIG. 4D) Fold change of each condition normalized to uncoated samples;

FIG. 5A, FIG. 5B, and FIG. 5C demonstrate that the fluorescence enhancement depends on the amount of matrix sprayed onto the fluorophore with a pneumatically driven sprayer, which is shown for CHCA matrix and rhodamine B (contained in Sharpie) (FIG. 5A). The more passes of CHCA were sprayed on rhodamine B, the more pronounced was the increase in fluorescence intensity (FIG. 5B), and fold change (FIG. 5C);

FIG. 6A, FIG. 6B, and FIG. 6C demonstrate co-crystallization of fluorophores with matrices. (FIG. 6A) From left to right, the columns demonstrate imaging results of matrix crystals, matrix and fluorophore cocrystals, and the boundary between the previous two crystals. Images were taken under polarized light. (FIG. 6B) Frames of crystals formation under different conditions, ranging from CHCA only, rhodamine B and CHCA co-crystallization, and FAD and CHCA co-crystallization. The last column demonstrates 20 biggest crystals selected under each condition for further analysis. (FIG. 6C) Intensity quantification of 20 crystals selected under each condition with respect to time;

FIG. 7A, FIG. 7B, FIG. 7C, FIG. 7D, FIG. 7E, and FIG. 7F demonstrate FluoMALDI pipeline performed with confocal microscope on healthy mouse brain samples half coated with CHCA. (FIG. 7A) whole half-coated brain samples at excitation wavelength of 405 nm and 488 nm. Two hippocampus horn regions are demonstrated in white (coated) and red (uncoated). (FIG. 7B) Comparison of hippocampus horn regions at different emission wavelengths excited at 405 nm. (FIG. 7C) Comparison of hippocampus horn regions at different emission wavelengths excited at 488 nm. (FIG. 7D) Optical and H&E scanning of the same tissue section used for confocal experiments. (FIG. 7E) MALDI MSI results of two identified lipid of the hippocampal region and their composite image at 5 μm lateral spatial resolution. (FIG. 7F) MALDI MSI spectra of selected two regions demonstrated in white in FIG. 7E;

FIG. 8A, FIG. 8B, and FIG. 8C demonstrate fluorescence enhancement of pink Sharpie samples coated with CHCA with different crystal sizes. (FIG. 8A) Optical scans and fluorescence images of pink Sharpie samples obtained under TRITC channel. (FIG. 8B) Quantification of raw fluorescence intensities of images taken under each condition, as demonstrated in part A. (FIG. 8C) MALDI MSI spectra of each condition. The labeled characteristic peak m/z⁺ 443.3 results from Rhodamine 6G, which is a main component in pink Sharpie samples;

FIG. 9A, FIG. 9B, and FIG. 9C demonstrate fluorescence enhancement of healthy mouse brain samples coated with CHCA with different crystal sizes. (FIG. 9A) Optical scans, MALDI MSI data, and fluorescence images of pink Sharpie samples obtained under GFP, TRITC, and DAPI channel. (FIG. 9B) Quantification of raw fluorescence intensities of images in TRITC and GFP channel taken under each condition, as demonstrated in part A. (FIG. 9C) MALDI MSI spectra of each condition;

FIG. 10 demonstrates fluorescence enhancement of exogenous fluorophores coated with CHCA. 6 common exogenous fluorophores (Rhodamine B, Fluorescein, Hoechst, Alexa Fluor 350, Alexa Fluor 488, Alexa Fluor 555) were sprayed with CHCA and the fluorescence images were taken to compare uncoated and coated samples with CHCA. Each fluorophore image was taken under TRITC, GFP, and DAPI channel;

FIG. 11 demonstrates fluorescence enhancement of endogenous fluorophores coated with CHCA. 4 common exogenous fluorophores (NADH, NADPH, FADH, PPIX) were sprayed with CHCA and the fluorescence images were taken to compare uncoated and coated samples with CHCA. Each fluorophore image was taken under TRITC, GFP, and DAPI channel;

FIG. 12A, FIG. 12B, FIG. 12C, and FIG. 12D demonstrate fluorescence enhancement of healthy mouse brain samples coated with various common matrices. (FIG. 12A) From left to right, the columns demonstrate imaging results from optical scans, fluorescence images using DAPI, GFP and TRITC channels, and MALDI data in negative ion mode focused on 888.75 m/z, which is the peak of PI 38:4. (FIG. 12B) Fluorescence intensities of samples coated with different matrices under TRITC channel. (FIG. 12C) Fluorescence intensities of samples coated with different matrices under GFP channel. (FIG. 12D) Fluorescence intensities of samples coated with different matrices under DAPI channel.

FIG. 13 demonstrates fluorescence enhancement of healthy mouse brain samples coated with different passes of CHCA;

FIG. 14A, FIG. 14B, and FIG. 14C demonstrate the presently disclosed FluoMALDI pipeline performed with confocal microscope on cerebellum of healthy mouse brain samples half coated with CHCA. (FIG. 14A) Optical scan and fluorescence image taken from the confocal microscope (FIG. 14B) H&E staining image of cerebellum with MALDI analysis region demonstrated in black. (FIG. 14C) MALDI data on demonstrated cerebellum region. 3 lipids were identified: PC (38:6), PC (36:1), LPA (14:0);

FIG. 15A, FIG. 15B, FIG. 15C, and FIG. 16D show confocal images of hippocampal horn region of healthy mouse brain samples half coated with CHCA. (FIG. 16A) Images taken at excitation wavelength=458 nm and emission wavelengths ranging from 503 nm to 566 nm. (FIG. 16B) Images taken at excitation wavelength=514 nm and emission wavelengths ranging from 539 nm to 602 nm. (FIG. 16C) Images taken at excitation wavelength=561 nm and emission wavelengths ranging from 575 nm to 638 nm. (FIG. 16D) Images taken at excitation wavelength=633 nm and emission wavelengths ranging from 611 nm to 674 nm;

FIG. 16A and FIG. 16B show microscopic images and full MALDI MSI spectra of selected two regions in the hippocampal horn area. (FIG. 16A) Optical scan and H&E staining images of full brain section utilized in confocal experiments. (FIG. 16B) MALDI MSI spectra of selected two regions with high intensity at m/z⁺ 478.33 and m/z⁺ 504.35, respectively;

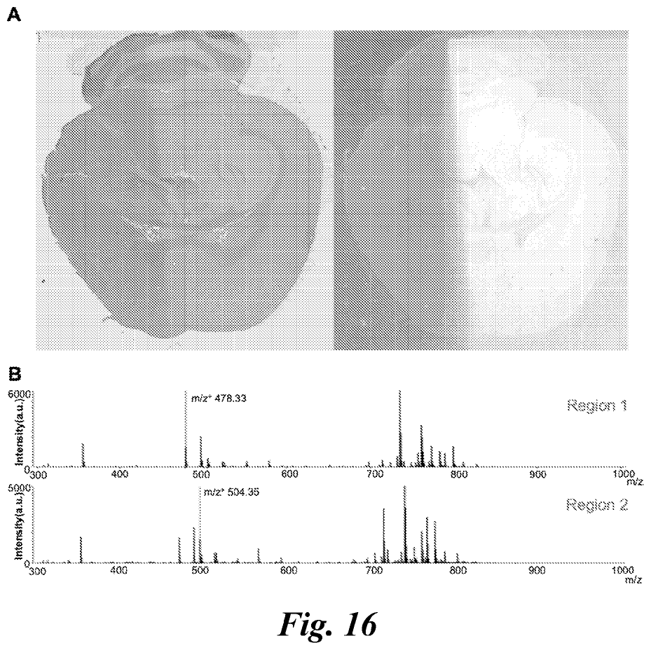

FIG. 17A and FIG. 17B show MS/MS spectra in positive ion mode of (FIG. 17A) peak and fragments of m/z⁺ 478.33 (FIG. 17B) peak and fragments of m/z⁺ 504.35. Chemical structures of characteristic fragments (boxed in red) are shown; and

FIG. 18A, FIG. 18B, FIG. 18C show MS/MS spectra in positive ion mode of (FIG. 18A) precursor and fragment peaks of standard Rhodamine B at m/z⁺ 443.122. (FIG. 18B) precursor and fragment peaks of pink Sharpie samples at m/z⁺ 443.122. (FIG. 18C) precursor and fragment peaks of pink Sharpie samples coated with MALDI matrix 1,5-Diaminonapthalene (DAN) at m/z⁺ 443.122.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

I. Enhanced Fluorescence Signal Through the Application of Aromatic Additives onto the Microscopy Sample for Standard Fluorescence, Fluorescence Microscopy and Combined Fluorescence Maldi Microscopy/Imaging

Identifying disease-specific molecular profiles requires an understanding of how biomolecules are altered in diseased tissue and the surrounding microenvironment. The development of such profiles ideally is achieved through an approach or a combination of approaches that measures biomolecular features in a high-throughput and spatially-resolved manner. Fluorescence spectroscopy, including fluorescence microscopy, and matrix-assisted laser desorption/ionization (MALDI) mass spectrometry, including MALDI imaging (MSI) are two techniques particularly well suited for the analysis of diseased tissue, or tissue suspected of being associated with a disease, condition, or disorder.

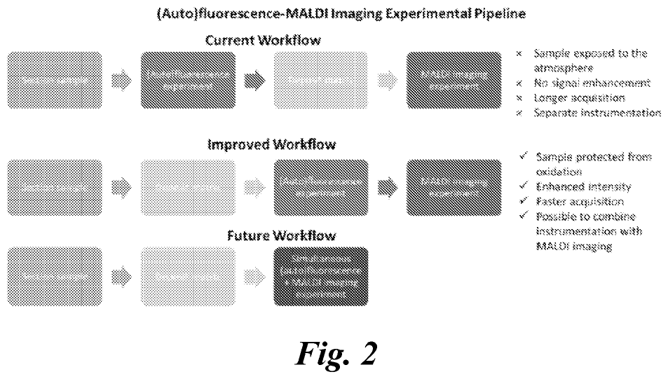

No workflows to date, however, have considered the addition of the MALDI matrix onto the sample for fluorescence detection. Workflows that combine MALDI and fluorescence have never demonstrated the ability to obtain autofluorescence results with the matrix already deposited. Currently, fluorescence imaging and MALDI-MS imaging are performed on different instruments, with separate workflows and separate samples. To combine both imaging modalities, current practices involve extra time to prepare and to image parallel samples. The presently disclosed workflow (see, e.g., FIG. 2) reduces the sampling time to one workday and minimizes sample degradation.

In some embodiments, the presently disclosed subject matter provides a method for enhancing a fluorescence intensity of a sample by adding an aromatic compound thereto. Aromatic compounds suitable for use with the presently disclosed methods include small organic molecules (less than about 300 Da), which have a strong UV absorbance in the 300 nm to 400 nm wavelength region.

In some embodiments, the presently disclosed subject matter provides a method for obtaining a fluorescence image of a sample as part of MALDI imaging workflow. The method comprising:
(a) providing a sample;
(b) depositing one or more aromatic additives onto the sample;
(c) obtaining a fluorescence image of the sample; and
(d) obtaining a MALDI mass spectrometry image of the sample.

In certain embodiments, the one or more aromatic additives have an absorbance ranging from about 300 nm to about 400 nm. In particular embodiments, the one or more aromatic comprise a matrix-assisted laser desorption/ionization (MALDI) matrix.

Several different classes of substances are suitable for use as MALDI-MSI matrices including small organic molecules, graphene, graphene oxide, nanoparticles, metal oxides, ionic liquids, and conjugated polymers. Small organic molecules are the most common MALDI-MSI matrices. Representative MALDI-MSI matrices include, but are not limited to,
alpha-cyano-4-hydroxycinnamic acid (CHCA), sinapinic acid (SA) ((2E)-3-(4-hydroxy-3,5-dimethoxyphenyl)prop-2-enoic acid), 2,5-dihydroxybenzoic acid (DHB), 3,4-dihydroxycinnamic acid (DHBA), 2-mercaptobenzothiazole (MBT), trans-3-indoleacrylic acid (IAA), 5-chloro-2-mercaptobenzothiazole (CMBT), 2-hydroxyphenylbenzoic acid (HPBA), 2-amino-4-methyl5-nitropyridine (2a4m5n), 2,6-diydroxyacetophenone (DHAP), trihydroxyacetophenone (THAP), ferulic acid (FA) ((2E)-3-(4-hydroxy-3-methoxyphenyl)prop-2-enoic acid), picolinic acid (PA) (pyridine-2-carboxylic acid), 3-hydroxy picolinic acid (HPA) (3-hydroxypyridine-2-carboxylic acid), caffeic acid (CA), 1,5-diaminonaphthalene (DAN), 9-aminoacridine (9-AA), norharmane (nH), nicotinic acid, pyrozinoic acid, vanillic acid, succinic acid, glycerol, urea, tris buffer (pH 7.3), other cinnamic acid derivatives, and combinations thereof.

Rationally designed and newly disclosed matrices for MALDI-MSI also are suitable for use with the presently disclosed subject matter including, but not limited to, 1,5-diaminonaphthalene, 4-phenyl-α-cyanocinnamic acid amide, alkylated 2,5-DHB, 1,8-di(piperidinyl)-naphthalene, 4,5-(bis(dimethylamino) naphthalen-1-yl)furan-2,5-dione, (E)-4-(2,5-dihydroxyphenyl) but-3-en-2-one (2,5-cDHA), 2-(methylamino)benzoic acid (2-COOH-NHMe), N-phenyl-2-naphthylamine (PNA), N-(1-naphthyl)ethylenediamine (NEDC), 3,4-dimethoxycinnamic acid (DMCA), 3-aminophthalhydrazide (3-APH), 2,3-dicyanohydroquinone (DCH), (2-[(2E)-3-(4-tert-butylphenyl)-2-methylprop-2-enylidene]malononitrile (DCTB), IR-780, 1,6-diphenyl-1,3,5-hexatriene (DPH), and 1,1′-binaphthyl-2,2′-diamine (BNDM). (See Zhou, Q., Fulop, A., and Hopf, C., Analytical and Bioanalytical Chemistry (2021) 413:2599-2617).

In addition to these small organic molecules, MALDI matrices often include agents to modulate ionization modes. MALDI matrices also can include one or more acid additives, including, but not limited to, H₃PO₄, HNO₃, H₂SO₄, HCl, trifluoroacetic acid (TFA), ammonium-based salts, sodium-based salts, lithium-based salts and potassium-based salts. In some embodiments, the acid additive is TFA. Acid additives serve as a proton donor to facilitate ionization of the sample. Basic MALDI matrices also have been used (depending on the mode of ionization, e.g., for positive or negative ions).

Because of their complex solubilities, matrix components are often dissolved in a solution comprising a mixture of water and organic solvents, thus solubilizing both hydrophobic and hydrophilic components. If sprayed, the solvent vaporizes, leaving a crystallized matrix with the sample embedded therein. The coated sample is then introduced into the mass spectrometer. In the next step a pulsed laser irradiates the sample, causing ablation and desorption of the sample and matrix material. Finally, the sample molecules are ionized by being protonated or deprotonated in the plume of ablated material and then they can be accelerated into the mass spectrometer for detection.

In certain embodiments, the MALDI matrix further comprises a solvent. In particular embodiments, the solvent is selected from acetonitrile, water, methanol, ethanol, propanol, acetone, chloroform, N,N-dimethylformamide, tetrahydrofuran and combinations thereof.

In certain embodiments, the MALDI matrix further comprises a counter ion or acid additive. In particular embodiments, the counter ion or acid additive is selected from H₃PO₄, HNO₃, H₂SO₄, HCl, trifluoroacetic acid (TFA), ammonium-based salts, sodium-based salts, lithium-based salts and potassium-based salts.

In certain embodiments, the depositing of the one or more aromatic additives onto the sample is accomplished by manual spraying, dry sieve deposition, robotic spraying, sublimation, or combinations thereof.

In certain embodiments, the method further comprises fluorescence microscopy, including filter-based lamps, confocal and two-photon fluorescence microscopy, among others.

In certain embodiments, the method further comprises obtaining a MALDI spectrum of the sample. In particular embodiments, the method further comprises MALDI imaging.

In certain embodiments, the sample is snap frozen or heat treated. In certain embodiments, the sample comprises a dried sample.

In certain embodiments, the method further comprises embedding the sample in an embedding medium, matrix, or resin. In particular embodiments, the embedding medium comprises an M-1 embedding medium, an optimal cutting temperature (OCT) embedding medium, agar, gelatin embedding medium, carboxymethyl cellulose (CMC) medium or ice. In certain embodiments, the sample is embedded in formalin-fixed paraffin (FFPE) matrix.

In certain embodiments, the method further comprises cryo-sectioning the sample. In particular embodiments, the sample is cryo-sectioned onto a microscopy glass slide, an indium tin oxide (ITO)-coated slide or other conductive slides, including but not limited to gold slides, aluminum oxide slides, steel plates, among others.

In certain embodiments, the method further comprises derivatizing the sample.

In certain embodiments, the method comprises analyzing one or more intact proteins. In certain embodiments, the method comprises analyzing one or more tryptic peptides. In certain embodiments, the method comprises analyzing one or more N-glycans, peptides, lipids, metabolites, drug molecules, and drug metabolites in a spatially resolved manner.

In certain embodiments the sample is selected from an organ, a tissue, or a cell. One of ordinary skill in the art would recognize that the presently disclosed methods are suitable for use with any tissue from any species.

In certain embodiments, a fluorescence signal of the sample with the one or more aromatic compounds deposited thereon is greater than a fluorescence signal of the sample without the one or more aromatic compounds deposited thereon.

In particular embodiments, the fluorescence is an autofluorescence intrinsic to the specimen. As used herein, the term “autofluorescence (AF)” refers to the natural emission of light, for example, in the UV-visible, near-IR spectral range, which occurs when a biological substrate is excited with light at suitable wavelength. Autofluorescence measurements do not require labeling the substrate with a fluorophore. In other embodiments, the fluorescence can arise from fluorescence from dyes or dye-conjugated reagents.

The presently disclosed methods are applicable for standard fluorescence imaging at cellular resolution, as well as combined fluorescence-matrix-assisted laser desorption/ionization (MALDI) mass spectrometry microscopy/imaging workflows. The presently disclosed methods offer a substantial improvement compared to the current technology and can be applied to fluorescence and MALDI microscopy and imaging fields (for example, single cells, fluorescently labelled proteins, and the like).

The presently disclosed subject matter addresses at least two problems, including an ability to detect molecules that were previously below the limit of detection of traditional fluorescence microscopy/imaging and protection from sample degradation. For example, in some embodiments, the signal enhancement was at least 5-fold, which suggests that signals near the detection threshold will now be detectable. This enhancement also translates to a lower overall fluorescence needed for strong signals and a faster acquisition time. This enhancement also means LESS dye needed for fluorescent detection. For subsequent MALDI imaging, there also will be less dye to confuse/obscure the MALDI spectrum.

Also, the presently disclosed subject matter provides an ability to combine the fluorescence-MALDI microscopy/imaging workflow with the MALDI matrix already applied, thereby improving overall sample quality and opening the door for novel instrumentation. It is known that certain molecular signals degrade over time with exposure to air, which will happen during the fluorescence microscopy/imaging step. Adding the matrix to conduct fluorescence will provide a protective barrier and enhanced signal.

Further, given that both techniques require the use of lasers in the UV wavelength range, the presently disclosed approach also opens the possibility to develop unique instrumentation for fluorescence+MALDI microscopy/imaging, rather than two separate machines that are currently required. We see the instrumentation as a strong possibility.

Potential uses of the presently disclosed methods include, but are not limited to, a fee-for-service for diagnostic purposes, should there be biomarkers that can now be detected on biopsies or other samples that can now be readily identified with this technique; novel instrumentation, including combined fluorescence/MSI instrumentation, since both techniques are laser based; and a pre-coated slide or cover comprising one or more signal-enhancing aromatic compounds.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### Enhanced Fluorescence Signal Through the Application of Aromatic Additives onto the Microscopy Sample for Standard Fluorescence, Fluorescence Microscopy and Combined Fluorescence MALDI Microscopy/Imaging

Aromatic compounds suitable for use with the presently disclosed methods include small organic molecules (less than about 300 Da), which have a strong UV absorbance in the 300 nm to 400 nm wavelength region, including, but not limited to, those provided in Table 1 herein below. The excitation and emission wavelength of matrices mentioned provided in Table 1 represent the TRITC (Rhodamine B) and GFP (Fluorescein) wavelengths. It is thought that the fluorescence enhancement is due in part to aromatic base-stacking arising from co-crystallization with matrix molecules during matrix application.

TABLE 1
Representative Matrices and Excitation/Emission Wavelengths

| Type | Compound | Excitation (nm) | Emission (nm) |
|---|---|---|---|
| Matrix | DAN | 330? | ~400 |
| | DHB | ~330 | 400-500 |
| | CHCA | 330 | 450 (monomer) |
| | | | 550 (dimer) |
| Fluorophore | Rhodamine B | 546 | 568 |
| | Fluorescein | 498 | 517 |

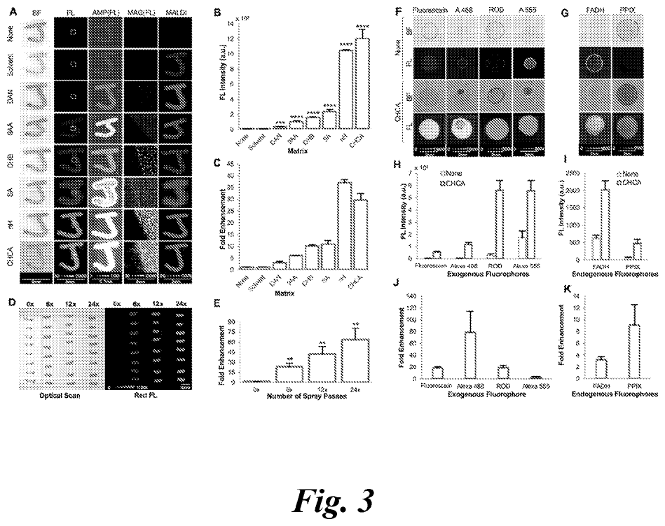

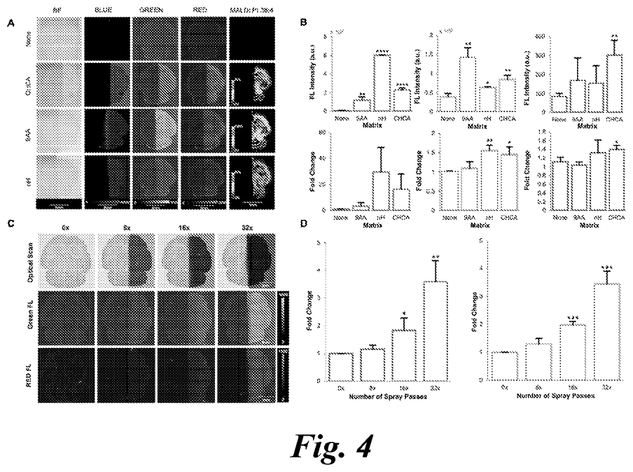

Overall fluorescence enhancement was observed for 300 nm to 600 nm excitation/500 nm to 700 nm emission experiments, on biological samples (see, for example, FIG. 3) and non-biological samples (see, for example, FIG. 4).

Although the mechanism of the observed enhancement is unclear, without wishing to be bound to any one particular theory, it is thought that the fluorescence enhancement is due in part to aromatic base-stacking arising from co-crystallization with matrix molecules during matrix application.

### Example 2

### Co-Crystallization Enhances Fluorescence and MALDI Signals Enabling FluoMALDI Tissue Microscopy

### 2.1 Overview

We report the discovery that co-crystallization of fluorophores with commonly used matrix-assisted laser desorption/ionization (MALDI) imaging matrices significantly enhances their fluorescence signal intensities of up to 30-fold, thereby also amplifying tissue autofluorescence contrast. This discovery facilitates FluoMALDI, the simultaneous measurement of fluorescence microscopy and MALDI imaging of the same sample or tissue section. Our approach combines the high spatial resolution, tissue autofluorescence detection, and fluorophore-tagging capabilities of fluorescence microscopy with the inherently multiplexed, versatile metabolic, lipidomic, and drug imaging capabilities of MALDI imaging. This new paradigm eliminates the requirement for two separate, consecutive tissue sections, overcoming previous significant limitations of not capturing the exact same cells in samples and necessitating complex data registration processes. In addition to the known ionization abilities of MALDI matrices, their co-crystallization with organic fluorophores significantly enhance the signal intensity in fluorescence microscopy, making possible applications that are limited by insufficient fluorescence signal intensity.

### 2.2 Background

Several million microscopic histology and cytology examinations are performed every year to diagnose cancers and other diseases. These rely mostly on hematoxylin-eosin stained tissue sections or cells. However, traditional tissue microscopy techniques frequently used for clinical diagnostics typically can only assess one or two protein markers in each tissue section. Recent developments in multiplexed imaging have allowed for visualization and quantification of more biomarkers in complex biological systems. For example, co-detection by indexing (CODEX) relies on DNA-conjugated antibodies for fluorescence imaging, enabling deep visualization of single-cell relationships in tissues and simultaneous detection of many markers in situ. Black et al., 2021. ChipCytometry can theoretically quantify unlimited protein biomarkers on the same sample. Jarosch et al., 2022. Other examples also include imaging mass cytometry (IMC), Kuett et al., 2022, and Multiplex Ion Beam Imaging (MIBI). Coskun et al., 2021; Keren et al., 2019. Both techniques allow simultaneous detection of dozens of metal-tagged antibodies at subcellular resolution and have recently advanced beyond the restrictions of 2D imaging into 3D format, allowing better visualization of microenvironmental heterogeneity and tissue organization. While these novel techniques can analyze many more biomarkers, they often require cycling procedures of staining and washing, which relies heavily on the integrity of the tissue. In addition, these staining experiments could not be achieved without prior knowledge of the targets of interest.

To overcome this difficulty, Matrix Assisted Laser Desorption/Ionization (MALDI) imaging is often incorporated in multimodal imaging approaches because of its inherently highly multiplexed nature. For example, Matrix Assisted Laser Desorption/Ionization (MALDI)-immunohistochemistry (IHC), Claes et al., 2023; Yagnik et al., 2021, is a new technology that relies on novel photocleavable mass-tags (PC-MTs) for labeling, enabling highly multiplexed IHC based on MALDI imaging. MALDI imaging has the great advantage of not requiring specific labels or knowledge of the tissue's biochemical composition, making it an excellent tool for molecular discovery and tissue surface analysis. In MALDI imaging, a tissue section is scanned with a laser in a gridlike fashion to generate a single mass spectrum of the molecular composition of any pixel within the scanned region. Claes et al., 2023; Yagnik et al., 2021; Aichler and Walch, 2015. MALDI imaging can detect a wide spectrum of analytes ranging from proteins, peptides, lipids, to small metabolites. Aichler and Walch, 2015; Basu et al., 2019. In addition, samples are coated with a thin layer of matrix crystals, which extract molecules of interest from the tissue to promote desorption and ionization by laser light. Due to the focus on their ionization abilities for better imaging results, Angerer et al., 2022; Leopold et al., 2018; Qiao and Lissel, 2021, published MALDI matrices studies have very limited information on optical properties. Even studies that mentioned such topics have mostly investigated the optical absorption of laser by matrices and the influence on experimental results. Qiao and Lissel, 2021; Robinson et al., 2018; Therefore, the optical and fluorescence properties of MALDI matrices remain to be explored.

MALDI imaging is often coupled with other high spatial resolution methods, such as fluorescence imaging, Blutke et al., 2020, and H&E staining, Deutshens et al., 2011, to provide additional morphological and compositional information from identical samples. Staining and microscopy have been widely utilized for clinical and research applications, providing a vast amount of morphological knowledge. Griffin and Treanor, 2017. Therefore, the addition of MALDI imaging, a rapidly growing imaging technique, often refers to existing knowledge from thin tissue analysis, providing a molecular dimension for integrated atlases. Patterson et al., 2018. The registration process of data is a critical step during multimodal imaging because the same tissue section could not be employed for MALDI imaging after the staining procedure. Many current methods still rely on manually aligning datasets based on the intensity patterns and fiducials, Chughtai et al., 2012; Scupakova et al., 2020, provided by users. These manual approaches often introduce human bias. Recent developments have improved the accuracy and speed of the registration process between datasets through computational approaches. For example, the Caprioli group developed novel workflows to register MALDI and wide field autofluorescence microscopy data by linking imaging mass spectrometry (IMS) pixels to its laser ablation pattern. Patterson et al., 2018. However, such methods still require rigorous time-consuming computation and external software.

In this example, we report the development of FluoMALDI workflow, a multimodal pipeline that integrates fluorescence and MALDI imaging while providing enhanced fluorescence signals and increased molecular information from the same tissue sample. FluoMALDI addresses several technical issues with multiplexing and circumvents limitations with current fluorescence and MALDI imaging workflows. In the FluoMALDI pipeline, we perform matrix coating before acquisition of both fluorescence and MALDI data. FluoMALDI simplifies and improves current workflows by: (1) producing great enhancement in overall fluorescence intensities; (2) allowing full experiment to be performed on the same tissue section; (3) achieving simple alignment between data sets through linear registration; (4) providing greater protection of sample for MALDI imaging through matrix coating. This novel pipeline can acquire highly multiplexed information on biological samples through the usage of MALDI imaging guided by morphological information from fluorescence imaging. More importantly, FluoMALDI only requires one sample throughout the whole workflow, thereby circumventing the technical challenges resulting from staining and complex data registration. Moreover, we also investigated in the fluorescence properties of MALDI matrices for the first time beyond the focus on ionization abilities in most published MALDI studies. Lastly, with the emergence of combined instrumentation of fluorescence and MALDI imaging, see JP2022182314A to Shimadzu Corp., 2022, our pipeline could further reduce the experimental time for such experiments in the future.

### 2.3 Results

### 2.3.1 FluoMALDI Imaging Pipeline

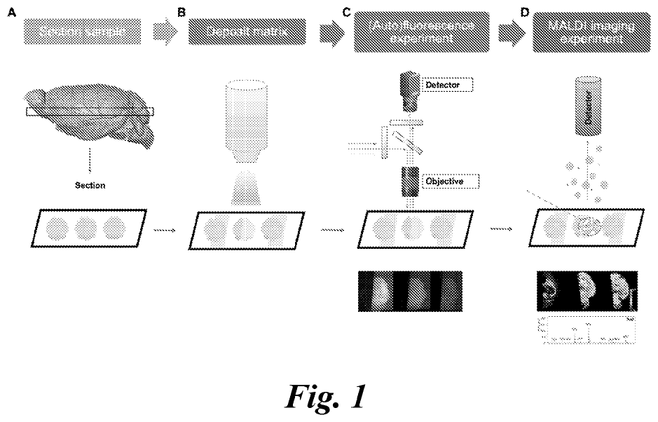

FIG. 1 provides an overview of our FluoMALDI multimodal imaging pipeline. First, biological samples are cryo-sectioned at 10 μm thickness and affixed to a conductive indium-tin oxide (ITO) microscopy slide. Next, the matrix of choice is deposited on samples to achieve a thin layer of coating. The choice of matrix and matrix thickness is optimized for downstream MALDI imaging experiments. Fluorescence microscopy is then performed using TRITC, GFP and/or DAPI filter sets on a regular widefield microscope or at specific wavelengths using a confocal microscope. Finally, MALDI imaging data was conducted with the desired parameters. The alignment of fluorescence and MALDI data and quantification of fluorescence signals could be performed by simple linear registration using ImageJ or other visual analysis software.

### 2.3.2 Fluorescence Signal Enhancement in Sharpie

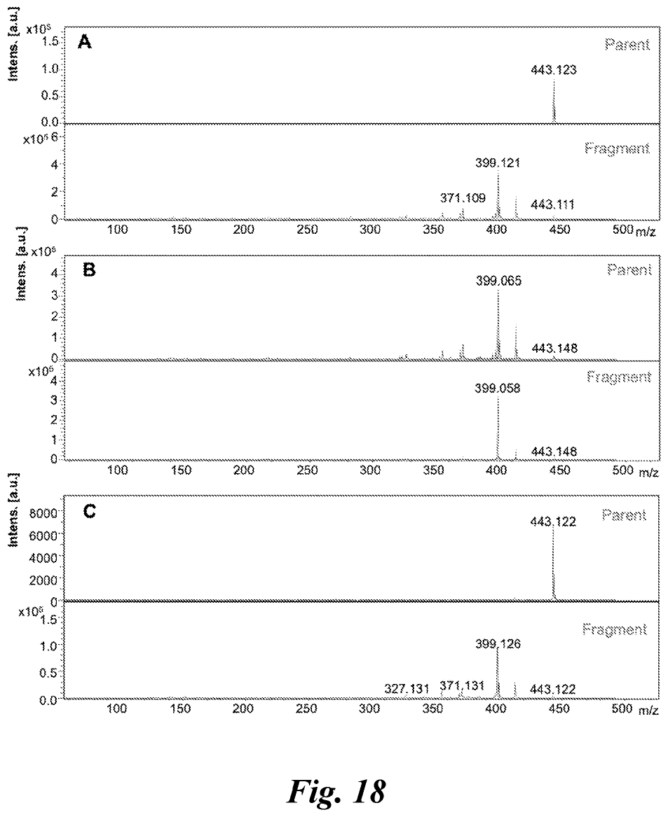

One key advantage of FluoMALDI is the discovery of enhanced fluorescence intensities when performing imaging with matrix coating on the samples. We first performed the full workflow on pink Sharpie samples, which contains exogenous fluorophore Rhodamine B (FIG. 18), to demonstrate such enhancement phenomenon. Fluorescence signal enhancement was observed from the pink Sharpie J's demonstrated in FIG. 3 with the addition of 6 common MALDI matrices. Representative fluorescence images of the Sharpie marks with each matrix coating and control (FIG. 3A) were acquired with the TRITC filter. Solvent alone did not contribute to any enhancement, while any matrix coating provided a drastic enhancement in fluorescence signal, with the greatest visual enhancement observed for nH and CHCA. Adjusted fluorescence enhancement from triplicate analyses indicated a minimum of 3.1 fold enhancement for Sharpie marks coated with matrix compared to the uncoated control Sharpie mark, with a maximum of 37 fold with nH (FIG. 3B and FIG. 3C). Adjusted fluorescence enhancement for CHCA was slightly lower at 29.7 fold due to the stronger matrix background. With the strong evidence of signal enhancement caused by matrix coating, we tested if the sprayed matrix density has a correlation with the intensity of enhancement. Horizontal lines drawn by the same pink Sharpie were sprayed with CHCA at different densities ranging from 0 mg/mm² to 0.0016 mg/mm² and imaged with the TRITC filter (FIG. 3D). The quantification demonstrates a positive correlation between matrix density and signal intensity (FIG. 3E).

### 2.3.3 Fluorescence Signal Enhancement in Exogenous and Endogenous Fluorophore Spots

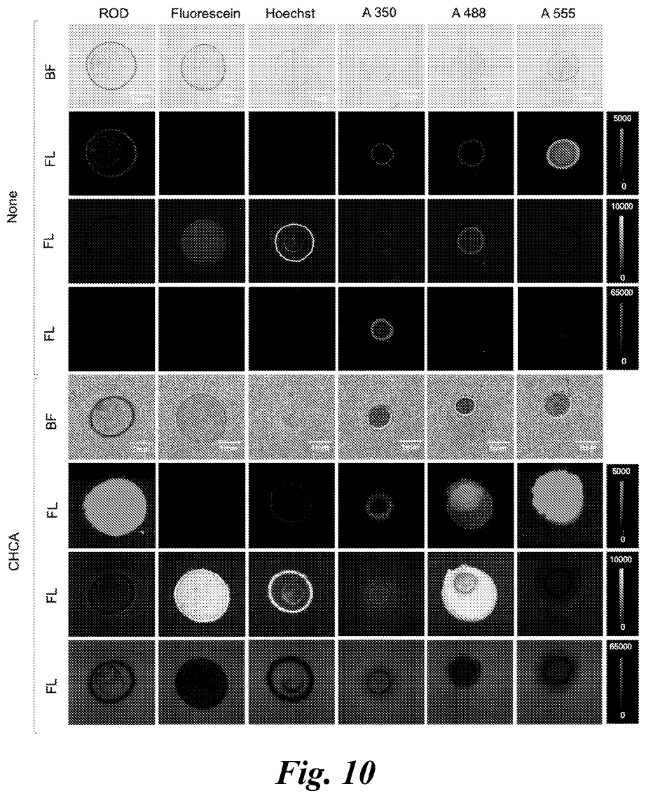

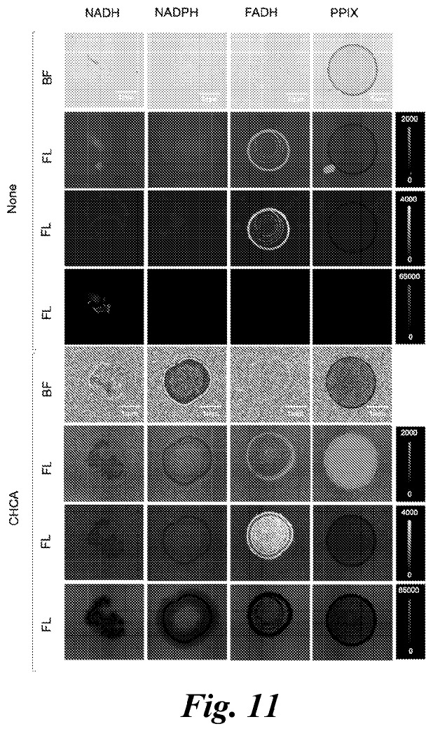

After demonstrating the discovery of enhanced fluorescence intensities with pink Sharpie samples containing Rhodamine B, we explored if such a phenomenon would be observed with a multitude of different fluorophores. CHCA coating was applied to each of the exogenous fluorophores with a concentration of 0.2 mg/mL (FIG. 3F). Representative images of Fluorescein and Alexa Fluor 488 were acquired using the GFP filter, while Rhodamine B and Alexa Flour 555 were imaged using the TRITC filter. All coated fluorophore spots demonstrated an increase of signal intensity compared to uncoated control spots, with Rhodamine B and Alexa Fluor 555 showing the highest signal around 60000 a.u. (FIG. 3H). The fold enhancement ranged from a minimum of 3.4 fold for Alexa Flour 555 to a maximum of 78.8 fold for Alexa Flour 488. In addition, we also investigated in the effect of matrix coating on endogenous fluorophores, such as flavin adenine dinucleotide (FAD) and Protoporphyrin IX (PPIX), both of which are frequently used biomarkers of energetic metabolism in biomedical research and clinical applications. Croce and Bottiroli, 2014. The endogenous fluorophore spots were also prepared and imaged under the same condition as the exogenous fluorophores, with FADH spot imaged using the GFP filter and PPIX imaged using the TRITC filter (FIG. 3G). Similar to the exogenous fluorophores, the endogenous fluorophores also demonstrated increase of signal intensities while coated with matrix, with 9.1 fold enhancement with PPIX and 3.2 fold enhancement with FADH (FIG. 3I and FIG. 3K). Other tested exogenous and endogenous fluorophores and images of each spot using TRITC, GFP and DAPI filters are reported in FIG. 10 and FIG. 11.

### 2.3.4 Fluorescence Signal Enhancement in Brain Tissue Samples

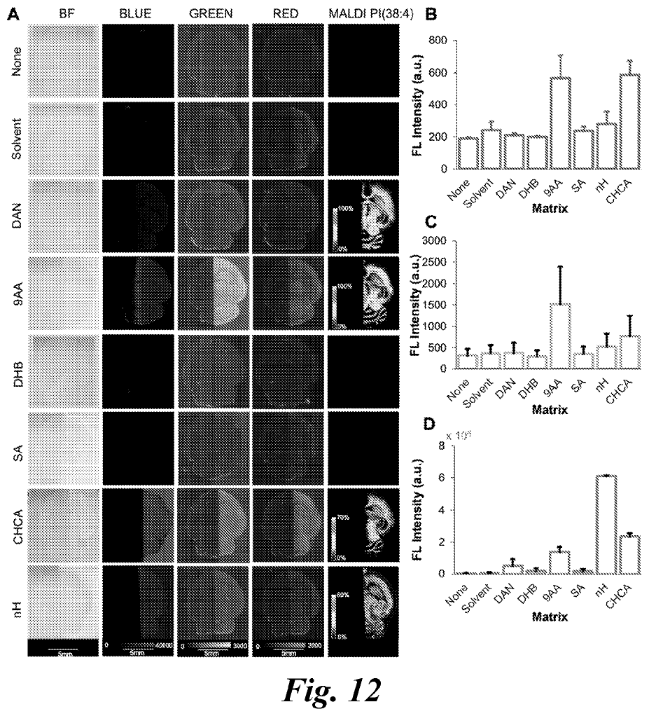

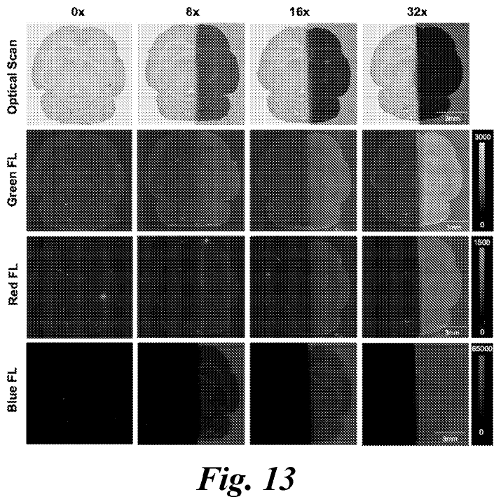

For brain tissue sections, autofluorescence without matrix spray was detected with all three channels, TRITC, DAPI and GFP, with varying intensities across the different histological regions of the brain (FIG. 4). The strongest autofluorescence without matrix coating was seen with the GFP channel. With matrix coating, all channels showed enhancements for the matrix-coated halves compared to the matrix-free control halves without losing histological specificity. Overall, CHCA, 9AA, and nH coated brain sections demonstrated the strongest adjusted fluorescence enhancement, though the enhancement varied by channel. Samples coated with nH are the clear winner in the DAPI channel at 29.6 fold enhancement, while CHCA showed strongest signals in the TRITC channel at 1.4 fold enhancement (FIG. 4B). 9AA demonstrated the strongest signal in terms of raw fluorescence intensity, but nH and CHCA showed bigger fold enhancement due to less background signal resulted from matrix crystals. Representative optical, autofluorescence and MALDI imaging results at m/z⁻ 885.55 (tentatively assigned PI 38:4) for these samples are shown in FIG. 4A. All other matrix-coated samples, DAN, DHB, and SA, are reported in FIG. 12. In addition, we also investigated in the correlation between matrix densities and signal enhancement intensities. The sample preparation was identical to the experiments using pink Sharpie samples. CHCA was again selected to be the matrix coating due to its wide usage for MALDI imaging, Leszyk, 2010, and the strong enhancement results in both Sharpie and brain samples. As the matrix coating increased from 0 mg/mm² to 0.0022 mg/mm², visual enhancement of fluorescence signal was observed (FIG. 4C). An exponential relationship was observed for the adjusted fluorescence enhancement and matrix thickness in the TRITC and GFP channel (FIG. 4D), while the DAPI channel suffered from intense matrix background fluorescence that drowned out the on-tissue autofluorescence (FIG. 13). Spatial integrity was preserved with increasing thickness. No accompanying MALDI images were acquired because the matrix thickness was no longer optimal for these experiments.

2.3.5 Co-Crystallization of Fluorophores with Matrices

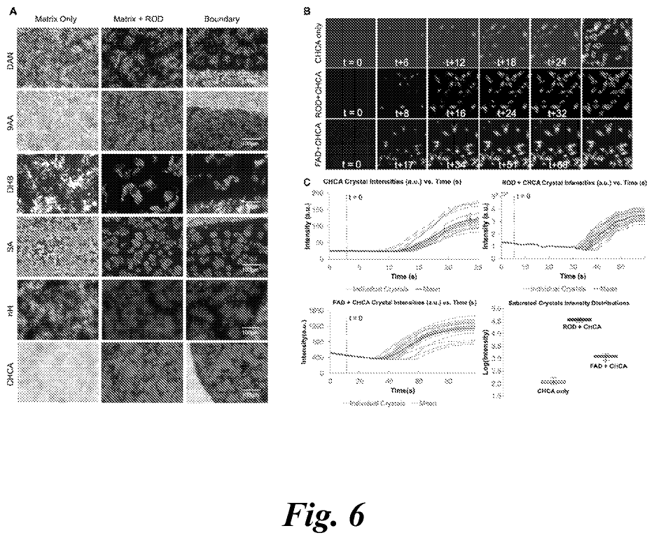

After demonstrating the discovered phenomenon of fluorescence enhancement, we inquired into potential mechanisms behind such a phenomenon. We started with observing morphological differences between crystals formed by matrix alone and by matrix with fluorophores. Images of coated Rhodamine B sharpie samples in FIG. 3 were taken using a polarized light at 10× zoom to observe the morphologies. Matrix formed on tissue were sparser and larger compared to those formed off tissue, with SA and DHB showing most noticeable difference and CHCA demonstrating the least visible change (FIG. 6). To demonstrate that there is indeed co-crystallization, we simulated the process through a time-course experiment where we spotted an equal volume of matrix solution into a spot of fluorophore solution and recorded the crystallization process (data not shown). For CHCA alone under the TRITC channel, the crystals formed irregular shapes that quickly gained intensity and leveled to an average raw intensity of 110. With rhodamine under the TRITC channel, the formed crystals incorporated the rhodamine molecules as it quickly gained intensity and ended with an approximate 300-fold increase compared to CHCA crystals alone. Finally, with FAD under the GFP channel, the crystals formed showed a more gradual increase in intensity up to an average 10-fold increase.

### 2.3.6 Fluorescence Enhancement in Laser-Based Confocal Microscopy Experiments

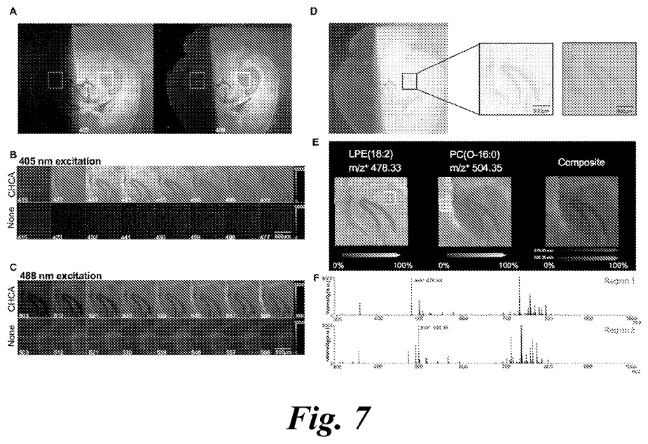

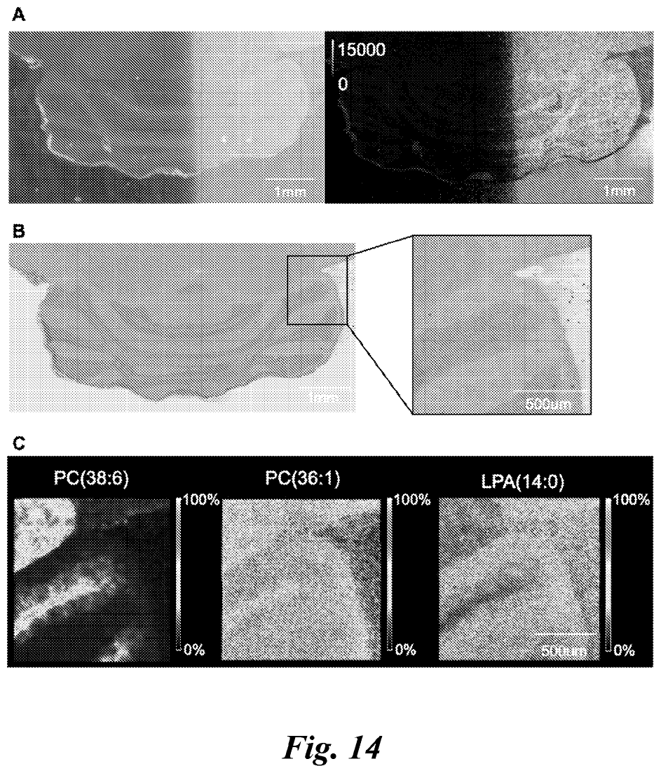

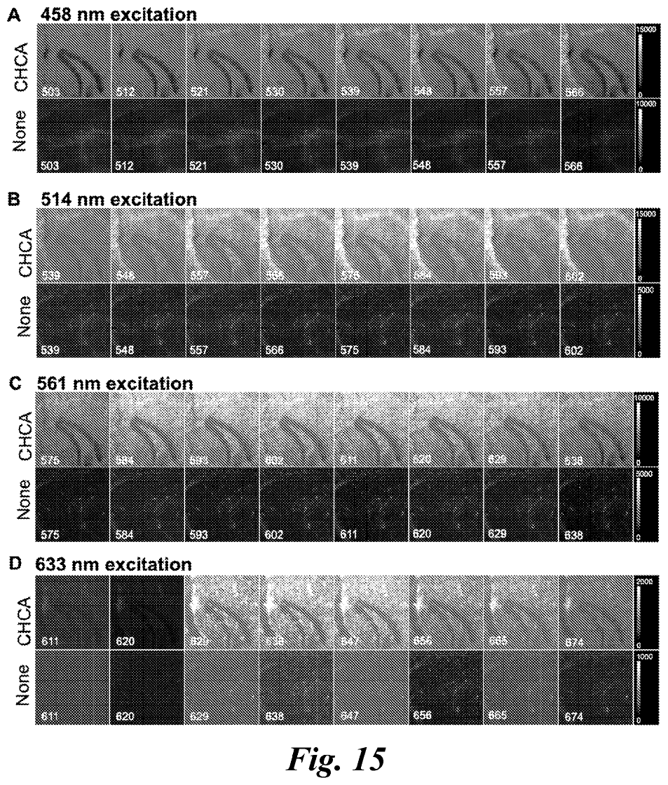

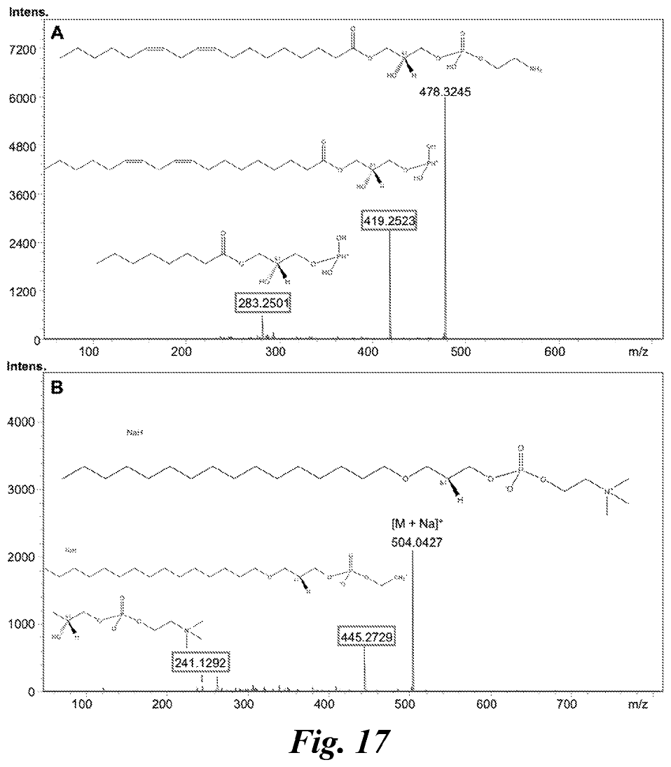

Fluorescence enhancements are still observable with the FluoMALDI workflow on laser-based confocal microscopes. Fluorescence images acquired on half-coated brain sections at 405 nm and 488 nm excitation showed appreciable fluorescence enhancement on the matrix-coated halves compared to the non-coated halves (FIG. 7A). The increased sensitivity allowed for the clear visualization of the hippocampal horn in the matrix-coated halves compared to the uncoated control. Of these two, 405 nm produced the greater fluorescence enhancement compared to 488 nm (FIG. 7B and FIG. 7C). Other wavelengths examined all produced weaker fluorescence enhancement (FIG. 15). MALDI MSI analysis was then performed on the same tissue section at 5 μm spatial resolution, with the molecular distributions of lipids and proteins clearly showing the layers of the hippocampal horn region. We identified two lipid peaks LPE (18:2) at m/z⁺ 478.33 and PC(O-16:0) at m/z⁺ 504.35 (FIG. 7E and FIG. 17). Additional imaging results on the cerebellum region at 5 μm spatial resolution are reported in FIG. 14. The full mass spectra of regions demonstrated in white boxes are shown in FIG. 7F. Optical scanning and H&E staining were performed after MALDI MSI experiments to complement structural information of the analyzed hippocampal horn region (FIG. 7D).

### 2.4 Discussion

Fluorescence imaging has long reigned as the standard approach for multiple types of spatial biological studies, such as protein location and associations, motility, and metabolism. Coling and Kachar, 2001; Combs and Shroff, 2017; Schouw et al., 2021. Its wide application can be owed to continuing advancements in fluorescence microscopy such as in fluorescent proteins, Matlashov et al., 2020; Shaner et al., 2005; Wiedenmann et al., 2009, confocal microscope, Elliott, 2020; Jonkman et al., 2020, and multi-photon microscopy, Goedeke et al., 2019; Ishii et al., 2022, that have made possible subcellular imaging on live and preserved samples for both targeted and untargeted studies. However, limitations including insufficient signal intensity, signal crosstalk, Orth et al., 2018, and lack of specific molecular information without tagging remain challenging. Mass spectrometry-based imaging techniques, specifically, matrix-assisted laser desorption/ionization (MALDI), overcomes some of these challenges as hundreds of molecules can be detected in one experiment on one tissue section with little knowledge a priori. Spatially, MALDI still lags behind that of fluorescence, however, as most commercial systems are limited to 5 μm spatial resolution with a noticeable drop in sensitivity due to the small pixel size.

In this example, we have developed the FluoMALDI workflow, a multimodal experimental pipeline that takes advantage of the complementary information from fluorescence and MALDI imaging, providing increased molecular information from the same tissue sample based on our discovery of enhanced fluorescence signals due to matrix application. One possible explanation behind the observed enhancement lies in the co-crystallization process, wherein analytes are embedded within micron-sized crystals of MALDI matrices during the matrix deposition step with the automatic sprayer. Specifically, on tissue fluorophores are believed to be incorporated into the matrix crystals, as indicated by the gain in intensities from crystals in the time-course experiments (FIG. 6C). Moreover, the crystal structural differences formed by various matrices suggest a possible relationship between the observed fluorescence enhancement and choice of matrix that warrant deeper investigation (FIG. 6A).

At first glance, the FluoMALDI pipeline appears to resemble previously described combined fluorescence-MALDI workflows. The counterintuitive innovation demonstrated in this article is the acquisition of fluorescence results after matrix coating, as conventional protocols call for fluorescence imaging prior to matrix deposition. Our results show the FluoMALDI pipeline improves the combined fluorescence and MALDI imaging workflow in the following three aspects: simplified registration, strong signal enhancement and greater quality of MALDI imaging. First, because sample preparation is complete prior to any imaging analyses, with appropriate care, no physical changes would occur between fluorescence and MALDI experiments. Therefore, combination of datasets only necessitates linear registration, which further simplifies alignment processes requiring computational methods. Patterson et al., 2018; Nikitina et al., 2020, FluoMALDI demonstrated greater clarity on histological features arising from fluorescence enhancement of endogenous compounds of the brain due to co-crystallization with matrices. For example, NAD(P)H and flavins are some possible contributors due to their studied roles in producing autofluorescence signal arising from the cell cytoplasm. Croce and Bottiroli, 2014. Finally, the FluoMALDI pipeline provides protection from degradation, as certain matrices, such as DAN, are known antioxidants that will extend the shelf life of biological samples, allowing for more extensive fluorescence experiments and flexibility during experimentation. With the protective matrix coating, fluorescence and MALDI experiments could be conducted on different days, whereas uncoated samples will require immediate matrix deposition after fluorescence imaging to avoid molecular degradation of molecules, especially lipids and metabolites. Patterson et al., 2014. In addition, less laser or light strength is needed during fluorescence experiments due to enhanced fluorescence, thereby further reducing possible degradation or damage on sensitive samples. Lastly, the FluoMALDI workflow has yet to demonstrate any signal loss in MALDI imaging results compared to conventional approaches (data not shown) and it does not hamper any further downstream pipelines that MALDI imaging is amenable to, e.g., H&E stains, IHC.

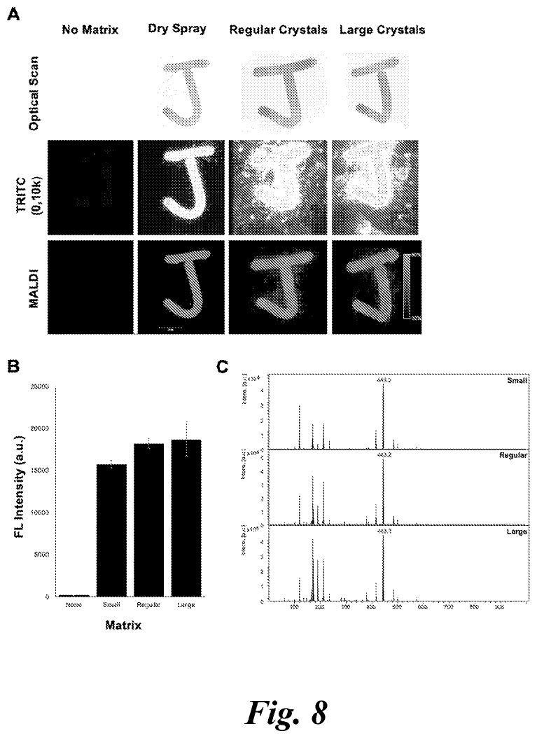

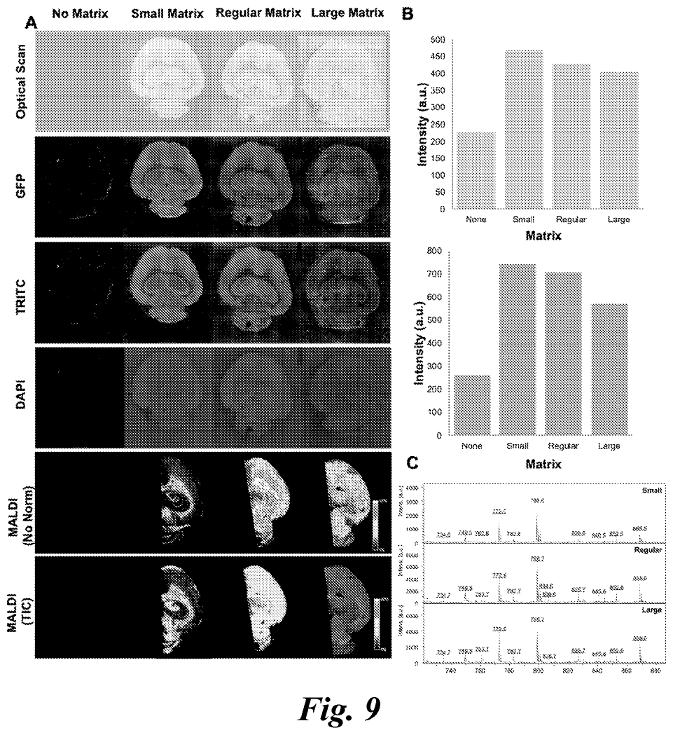

While FluoMALDI offers many advantages over the conventional combined fluorescence-MALDI protocol, it still suffers from several limitations. For one, samples are to be sectioned or dried, removing possibilities of adaptation for live imaging experiments. Such restraints are inherent in the protocol and cannot be easily overcome. Second, the fluorescence spatial quality can be reduced with the MALDI matrices, as the matrix deposition process introduces the chance for analyte delocalization, which is a common issue during wet matrix application, Anderson et al., 2015; Fournelle et al., 2020; Scupakova et al., 2020, that could affect special resolution (FIG. 8 and FIG. 9). This issue may limit sub-micron imaging capabilities but could be avoided with more advanced matrix deposition approaches such as sublimation or more volatile solvent systems that greatly reduce matrix sizes. Finally, one obvious limitation is the continual need for two instrumentations, one for fluorescence and another for MALDI imaging, which could result in operational error that may lead to ruined samples. One potential avenue worth investigation is the combination of fluorescence with MALDI imaging that can be achieved either with conventional microscopes and broad UV lamps, or with more focused lasers. These possibilities are not out of the question. iMScope from Shimadzu offers a combined brightfield microscope and MALDI system, while Bruker has a MALDI system with a secondary laser for post-ionization. As the research community seeks to increase molecular information at the spatial level, FluoMALDI is a powerful tool enabling researchers to conduct research in this direction.

### 2.5 Methods

### 2.5.1 Chemicals and Reagents

Unless otherwise noted, all solvents and trifluoroacetic acid (TFA) were purchased from Sigma Aldrich (St. Louis, MO). 1,5-Diaminonapthalene (DAN), α-Cyano-4-hydroxycinnamic acid (CHCA), 2,5-Dihydroxybenzoic acid (DHB), norharmane (nH), 9-aminoacridine (9AA) and sinapic acid (SA) MALDI matrices were purchased from Sigma Aldrich (St. Louis, MO). NADH disodium, NADPH disodium, FADH disodium, Fluorescein, Rhodamine B fluorophores were also acquired from Sigma Aldrich (St. Louis, MO), and Hoechst 33342 was obtained from Thermo Fisher (Waltham, MA). Pink Sharpie was purchased from a local retail store. Solvents were all MS grades.

### 2.5.2 Animal Tissue Samples

All animal experiments were approved by the Institutional Animal Care and Use Committee (IACUC) of the Johns Hopkins University School of Medicine, which is fully accredited by the American Association for the Accreditation of Laboratory Animal care (AAALAC). Thirteen-week-old healthy mice were sacrificed. All mice were bred in the Research Animal Resources (RAR) at the Johns Hopkins University School of Medicine. Their brains were immediately dissected, flash frozen on liquid nitrogen and stored at −80° C. until experiments.

### 2.5.3 Fluorophore Sample Preparation

The pink Sharpie was used to scribe a series of the letter “J” approximately 5 mm by 5 mm across two conductive indium-tin-oxide (ITO) slides (Delta Technologies, Loveland, CO). The most common MALDI matrices were spray deposited onto the J's with an M5 sprayer from HTX Technologies (Chapel Hill, NC). Specifically, 50 mM of matrix at 0.05 mL/min flow rate, 10 psi, 1800 mm/min track velocity, 60° C., 3 L/min gas flow rate, 40 mm nozzle height, and 0s drying time was sprayed to achieve matrix density of 0.0016. The density is calculated based on the matrix density equation provided in the HTX sprayer manual. cmu.edu/chemistry/facilities/cma/pdfs/htx-spray_user_manual.pdf. All MALDI matrices solutions were prepared in 50% acetonitrile/water+0.1% Trifluoroacetic acid. To study the effects of matrix density on fluorescence intensity, lines of pink Sharpie of approximately 3 mm in length and 1 mm in thickness were transcribed across the ITO slide, allowed to dry under ambient conditions, and sprayed with 50 mM of CHCA with the same spray protocols as mentioned above. The number of passes were increased to achieve a density of 0.0004, 0.0008, 0.0016. Finally, the co-crystallization experiment shown in FIG. 6 involved mixing an equal amount of 0.1 mM solution of the fluorophores and the CHCA matrix solution, both made in 50% acetonitrile/water+0.1% TFA, onto a glass substrate at the microscope. All other fluorophore solutions for the remainder of the project were dissolved in water.

### 2.5.4 Biological Sample Preparation

Horizontal mouse brain sections were acquired at 10 μm thickness using a CM1860 UV Cryostat from Leica Biosystems (Wetzlar, Germany) and thaw mounted on ITO slides from Delta Technologies (Loveland, CO). The sections were kept in the −80° C. freezer until matrix deposition. Matrices were deposited with the sample spray parameters as described for the Pink Sharpie J's. During the spray, half of each brain section was covered by aluminum foil to remain matrix free and serve as controls. After matrix deposition, all brain slides were wrapped with aluminum foil, vacuum sealed and stored at −20° C. until fluorescence and MALDI imaging experiments. To minimize water condensation and resulting delocalization, samples were allowed to reach room temperature prior to breaking the vacuum for the downstream analyses.

### 2.5.5 Histology

For kidney and brain tissue sections, samples were submerged in ethanol for 24 hours to wash off MALDI matrices, followed by hematoxylin and eosin (H&E) staining conducted on the same tissue section using a standard protocol with Mayer's hematoxylin solution and aqueous Eosin Y solution from Sigma Aldrich (St. Louis, MO). H&E-stained slides were imaged at 40× magnification using a NanoZoomer S210 Digital slide scanner (Hamamatsu Photonics, Shizuoka, Japan). The H&E-stained tissue sections were visualized and exported from Aperio ImageScope (v 12.4.6, Leica Biosystems, Deer Park, IL).

### 2.5.6 Brightfield and Fluorescence Imaging

All fluorescence images were captured with an ImageXpress Micro High-Content Imager (Molecular Devices, San Jose, CA) using 4× objective. DAPI (excitation=405/20 nm, emission=452/45 nm), TRITC (excitation=543 nm, emission=593 nm), and GFP (excitation=472 nm, emission=520 nm) filters were chosen to capture possible enhancements at different excitation and emission wavelengths. Exposure times for all sharpie images were kept at 20 ms, and at 150 ms for all tissue images. Brightfield images of all slides were obtained using a Epson Perfection V850 Pro slide scanner in 24-bit color mode at 3200 dpi or higher with backlight correction and autoexposure. FIG. 6 videos were acquired using an Olympus microscope with exposure time at 100 ms. The TRITC filter was used for Rhodamine B and CHCA films, and the GFP filter was used for FAD films.

### 2.5.7 Polarized Light Microscopy

Polarized light microscopic images were acquired using the Olympus color station. 10× objective was employed for all images.

### 2.5.8 Confocal Microscopy

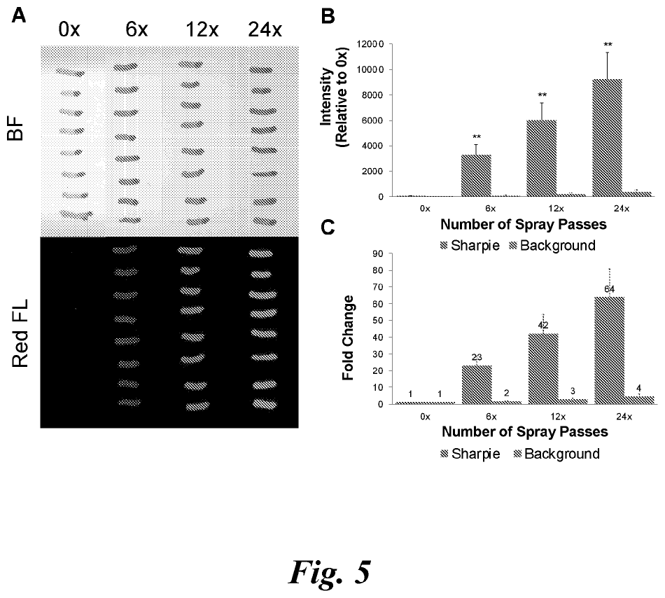

All confocal images were taken with a Zeiss AxioObserver with 780-Quasar confocal module (Zeiss Inc, Dublin, CA) with a 10× objective. There excitation wavelengths ranged from 405 nm to 633 nm, and the emission wavelengths ranged from 415 nm to 703 nm. The images were exported and changed to the Fire color scheme in ImageJ to allow better visualization of the intensity differences. While images with excitation wavelength ranging from 405 nm to 633 nm were taken, only data from excitation wavelengths at 405 nm and 488 nm are reported in FIG. 5 for simplicity of layout and the strong fluorescence intensities produced. The rest of the results can be found in FIG. 15.

### 2.5.9 MALDI Imaging Mass Spectrometry

All MALDI imaging except experiment from FIG. 7 was performed with a Bruker rapifleX TOF/TOF (Bruker Daltonics, Bremen, Germany) system equipped with a 10 kHz smartbeam 3D Nd:YAG laser at 355 nm. flexControl (v 4.0) was used to control the instrument and optimize the acquisition parameters, while flexImaging (v 5.0) was used to set up the imaging runs and select the regions of interest. All images were acquired from m/z 600-1000 in negative reflectron ion mode at 50 μm raster width with 150 shots per pixel using the single laser beam scan mode, 46 μm scan range and 10 KHz frequency. The extraction voltage was set to −20 kV, lens voltage of −11.3 kV, reflectron voltages at −20.86 kV, −1.085 kV and −8.6 kV, with a delay time of 100 ns, while the laser fluence was optimized for each matrix. Prior to data acquisition, height adjustment (target profile generation), laser focus tuning, and external mass calibration with red phosphorus were conducted to achieve a mass error <5 PPM. For Sharpie® drawings data were acquired in positive ion reflectron mode at 200 laser shots per pixel with a raster width of 100 μm in m/z 200-500 in beam scan mode. Brain tissue sections were imaged at 50 μm raster width with dual polarity of negative ion mode from m/z 0-1000 followed by positive ion mode from m/z 0-1700.

### 2.5.10 Image Processing and Data Analysis

Fluorescence images were exported to FIJI ImageJ (v 1.53f51) for visualization and quantification. Flat Fielding was performed first to avoid grid lines on images with lower intensities during tile stitching. All fluorescence images were also subtracted by a dark frame signal of 100 a.u. before any quantification. Measurements on tissue and off tissue were both taken to calculate the adjusted fluorescence enhancement, which is the fold change between the matrix-coated and uncoated sample acquired with the same microscope settings after normalizing the raw intensities with the matrix fluorescence background. FIG. 7 videos were first exported to SlideBook for visualization, then converted to be analyzed in FIJI ImageJ. Auto selection of crystals were performed using an 80% threshold for all images. The selected crystals were created into a mask and saved as multiple ROIs. MultiMeasure function was then employed to acquire intensities of each crystal at each time frame. MALDI imaging data was visualized in flexImaging (v 5.0) with TIC normalization.

### 2.5.11 MALDI MSI Data Processing and Analysis

The MALDI MSI data processing varied based on instrument used. For experiments conducted on Bruker rapifleX TOF/TOF, MALDI MSI data were directly exported from flexImaging (v 5.0, Bruker Daltonics) with total ion current (TIC) normalization. For imaging experiment in FIG. 6, MALDI MSI data were imported into SCiLS Lab (v 2021b, Bruker Daltonics) and segmentation analysis was conducted on TIC normalized data through the software's segmentation pipeline, which conducts peak picking and peak alignment and smoothing prior to segmentation using the bisecting k-means in combination with correlation distance. Segmentation maps were generated and exported from SCiLS Lab. Individual representative m/z features from each segment were identified from the segmentation analysis and the corresponding results were then visualized in flexImaging.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Black, S. et al. CODEX multiplexed tissue imaging with DNA-conjugated antibodies. Nat Protoc 16, 3802-3835 (2021).
Jarosch, S., Kohlen, J., Wagner, S., D'Ippolito, E. & Busch, D. H. ChipCytometry for multiplexed detection of protein and mRNA markers on human FFPE tissue samples. STAR Protoc 3, 101374 (2022).
Kuett, L. et al. Three-dimensional imaging mass cytometry for highly multiplexed molecular and cellular mapping of tissues and the tumor microenvironment. Nat Cancer 3, 122-133 (2022).
Coskun, A. F. et al. Nanoscopic subcellular imaging enabled by ion beam tomography. Nat Commun 12, 789 (2021).
Keren, L. et al. MIBI-TOF: A multiplexed imaging platform relates cellular phenotypes and tissue structure. Sci Adv 5, eaax5851 (2019).
Claes, B. S. R. et al. MALDI-IHC-Guided In-Depth Spatial Proteomics: Targeted and Untargeted MSI Combined. Anal Chem 95, 2329-2338 (2023).
Yagnik, G., Liu, Z., Rothschild, K. J. & Lim, M. J. Highly Multiplexed Immunohistochemical MALDI-MS Imaging of Biomarkers in Tissues. J Am Soc Mass Spectrom 32, 977-988 (2021).
Aichler, M. & Walch, A. MALDI Imaging mass spectrometry: current frontiers and perspectives in pathology research and practice. Lab Invest 95, 422-431 (2015).
Basu, S. S. et al. Rapid MALDI mass spectrometry imaging for surgical pathology. NPJ Precis Oncol 3, 17 (2019).
Angerer, T. B., Bour, J., Biagi, J. L., Moskovets, E. & Frache, G. Evaluation of 6 MALDI-Matrices for 10 mum Lipid Imaging and On-Tissue MSn with AP-MALDI-Orbitrap. J Am Soc Mass Spectrom 33, 760-771 (2022).
Leopold, J., Popkova, Y., Engel, K. M. & Schiller, J. Recent Developments of Useful MALDI Matrices for the Mass Spectrometric Characterization of Lipids. Biomolecules 8 (2018)
Qiao, Z. & Lissel, F. MALDI Matrices for the Analysis of Low Molecular Weight Compounds: Rational Design, Challenges and Perspectives. Chem Asian J 16, 868-878 (2021).
Robinson, K. N., Steven, R. T. & Bunch, J. Matrix Optical Absorption in UV-MALDI MS. J Am Soc Mass Spectrom 29, 501-511 (2018).
Blutke, A. et al. Light sheet fluorescence microscopy guided MALDI-imaging mass spectrometry of cleared tissue samples. Sci Rep 10, 14461 (2020).
Deutskens, F., Yang, J. & Caprioli, R. M. High spatial resolution imaging mass spectrometry and classical histology on a single tissue section. J Mass Spectrom 46, 568-571 (2011).
Griffin, J. & Treanor, D. Digital pathology in clinical use: where are we now and what is holding us back? Histopathology 70, 134-145 (2017).
Patterson, N. H., Tuck, M., Van de Plas, R. & Caprioli, R. M. Advanced Registration and Analysis of MALDI Imaging Mass Spectrometry Measurements through Autofluorescence Microscopy. Anal Chem 90, 12395-12403 (2018).
Chughtai, K. et al. Fiducial markers for combined 3-dimensional mass spectrometric and optical tissue imaging. Anal Chem 84, 1817-1823 (2012).
Scupakova, K., Dewez, F., Walch, A. K., Heeren, R. M. A. & Balluff, B. Morphometric Cell Classification for Single-Cell MALDI-Mass Spectrometry Imaging. Angew Chem Int Ed Engl 59, 17447-17450 (2020).
Croce, A. C. & Bottiroli, G. Autofluorescence spectroscopy and imaging: a tool for biomedical research and diagnosis. Eur J Histochem 58, 2461 (2014).
Leszyk, J. D. Evaluation of the new MALDI matrix 4-chloro-alpha-cyanocinnamic acid. J Biomol Tech 21, 81-91 (2010).
Coling, D. & Kachar, B. Principles and application of fluorescence microscopy. Curr Protoc Mol Biol Chapter 14, Unit 14 10 (2001).
Combs, C. A. & Shroff, H. Fluorescence Microscopy: A Concise Guide to Current Imaging Methods. Curr Protoc Neurosci 79, 2 1 1-2 1 25 (2017).
Schouw, H. M. et al. Targeted optical fluorescence imaging: a meta-narrative review and future perspectives. Eur J Nucl Med Mol Imaging 48, 4272-4292 (2021).
Matlashov, M. E. et al. A set of monomeric near-infrared fluorescent proteins for multicolor imaging across scales. Nat Commun 11, 239 (2020).
Shaner, N. C., Steinbach, P. A. & Tsien, R. Y. A guide to choosing fluorescent proteins. Nat Methods 2, 905-909 (2005).
Wiedenmann, J., Oswald, F. & Nienhaus, G. U. Fluorescent proteins for live cell imaging: opportunities, limitations, and challenges. IUBMB Life 61, 1029-1042 (2009).
Elliott, A. D. Confocal Microscopy: Principles and Modern Practices. Curr Protoc Cytom 92, e68 (2020).
Jonkman, J., Brown, C. M., Wright, G. D., Anderson, K. I. & North, A. J. Tutorial: guidance for quantitative confocal microscopy. Nat Protoc 15, 1585-1611 (2020).
Goedeke, J. et al. Multiphoton microscopy in the diagnostic assessment of pediatric solid tissue in comparison to conventional histopathology: results of the first international online interobserver trial. Cancer Manag Res 11, 3655-3667 (2019).
Ishii, H., Otomo, K., Takahashi, T., Yamaguchi, K. & Nemoto, T. Focusing new light on brain functions: multiphoton microscopy for deep and super-resolution imaging. Neurosci Res 179, 24-30 (2022).
Orth, A. et al. Super-multiplexed fluorescence microscopy via photostability contrast. Biomed Opt Express 9, 2943-2954 (2018).
Nikitina, A. et al. A Co-registration Pipeline for Multimodal MALDI and Confocal Imaging Analysis of Stem Cell Colonies. J Am Soc Mass Spectrom 31, 986-989 (2020).
Yong, D. & Ding, D. Lasing with cell-endogenous fluorophores: parameters and conditions. Sci Rep 7, 13569 (2017).
Patterson, N. H., Thomas, A. & Chaurand, P. Monitoring time-dependent degradation of phospholipids in sectioned tissues by MALDI imaging mass spectrometry. J Mass Spectrom 49, 622-627 (2014).
Anderson, D. M. et al. A method to prevent protein delocalization in imaging mass spectrometry of non-adherent tissues: application to small vertebrate lens imaging. Anal Bioanal Chem 407, 2311-2320 (2015).
Fournelle, F., Yang, E., Dufresne, M. & Chaurand, P. Minimizing Visceral Fat Delocalization on Tissue Sections with Porous Aluminum Oxide Slides for Imaging Mass Spectrometry. Anal Chem 92, 5158-5167 (2020).
Scupakova, K. et al. Cellular resolution in clinical MALDI mass spectrometry imaging: the latest advancements and current challenges. Clin Chem Lab Med 58, 914-929 (2020).
JP2022182314A for Matrix-Assisted Laser Desorption Ionization Mass Spectroscopy Device and Method, to Shimadzu Corp., published Dec. 8, 2022.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for obtaining a fluorescence image of a sample, the method comprising:
(a) providing a sample;
(b) depositing one or more aromatic additives onto the sample; and
(c) obtaining a fluorescence image of the sample.

2. The method of claim 1, wherein the one or more aromatic additives have an absorbance ranging from about 200 nm to about 500 nm.

3. The method of claim 1, wherein the one or more aromatic additives comprise a matrix-assisted laser desorption/ionization (MALDI) matrix.

4. The method of claim 3, wherein the MALDI matrix is selected from alpha-cyano-4-hydroxycinnamic acid (CHCA), sinapinic acid (SA) ((2E)-3-(4-hydroxy-3,5-dimethoxyphenyl)prop-2-enoic acid), 2,5-dihydroxybenzoic acid (DHB), 3,4-dihydroxycinnamic acid (DHBA), 2-mercaptobenzothiazole (MBT), trans-3-indoleacrylic acid (IAA), 5-chloro-2-mercaptobenzothiazole (CMBT), 2-hydroxyphenylbenzoic acid (HPBA), 2-amino-4-methyl5-nitropyridine (2a4m5n), 2,6-diydroxyacetophenone (DHAP), trihydroxyacetophenone (THAP), ferulic acid (FA) ((2E)-3-(4-hydroxy-3-methoxyphenyl)prop-2-enoic acid), picolinic acid (PA) (pyridine-2-carboxylic acid), 3-hydroxy picolinic acid (HPA) (3-hydroxypyridine-2-carboxylic acid), caffeic acid (CA), 1,5-diaminonaphthalene (DAN), 9-aminoacridine (9-AA), norharmane (nH), nicotinic acid, pyrozinoic acid, vanillic acid, succinic acid, glycerol, urea, tris buffer (pH 7.3), other cinnamic acid derivatives, and combinations thereof.

5. The method of claim 3, wherein a solution comprises a solvent and the MALDI matrix to be deposited.

6. The method of claim 5, wherein the solvent is selected from acetonitrile, water, methanol, ethanol, propanol, acetone, chloroform, N,N-dimethylformamide, tetrahydrofuran and combinations thereof.

7. The method of claim 5, wherein the solution comprising the MALDI matrix further comprises a counter ion, acid additive or salt additive.

8. The method of claim 7, wherein the counter ion, acid additive or salt additive is selected from H₃PO₄, HNO₃, H₂SO₄, HCl, trifluoroacetic acid (TFA), ammonium-based salts, sodium-based salts, potassium-based salts and lithium-based salts.

9. The method of claim 1, wherein the depositing of the one or more aromatic additives onto the sample is accomplished by manual spraying, manual sieve deposition, robotic spraying, sublimation, or combinations thereof.

10. The method of claim 1, further comprising at least one of: fluorescence microscopy; obtaining a MALDI mass spectrum of the sample; and/or MALDI imaging.

11.-12. (canceled)

13. The method of claim 1, wherein the sample is snap frozen, heat treated, or comprises a dried sample.

14. (canceled)

15. The method of claim 1, further comprising embedding the sample in an embedding medium, matrix, or resin.

16. (canceled)

17. The method of claim 1, wherein the sample is embedded in formalin-fixed paraffin (FFPE) matrix.

18. The method of claim 1, further comprising cryo-sectioning the sample.

19. The method of claim 18, wherein the sample is cryo-sectioned onto a microscopy glass slide, an indium tin oxide (ITO)-coated slide or other conductive slides, including but not limited to gold slides, aluminum oxide slides, steel plates, among others.

20. The method of claim 1, further comprising derivatizing the sample.

21. The method of claim 1, comprising analyzing: one or more intact proteins; one or more tryptic peptides; or one or more N-glycans, peptides, lipids, metabolites, drug molecules, and drug metabolites in a spatially resolved manner.

22.-23. (canceled)

24. The method of claim 1, wherein the sample is selected from an organ, a tissue, or a cell.

25. The method of claim 1, wherein a fluorescence signal of the sample with the one or more aromatic compounds deposited thereon is greater than a fluorescence signal of the sample without the one or more aromatic compounds deposited thereon.

26. The method of claim 1, wherein the fluorescence is an autofluorescence.
